(19) United States
(12) Patent Application Publication
(10) Pub. No.: US 2016/0199221 A1
(43) Pub. Date: Jul. 14, 2016

(54) All Purpose Hot/Cold Therapy Gel Pack

(71) Applicants: Mufaddal Bootwala, Plano, TX (US); Insiya Bootwala, Plano, TX (US)

(72) Inventors: Mufaddal Bootwala, Plano, TX (US); Insiya Bootwala, Plano, TX (US)

(21) Appl. No.: 14/983,219

(22) Filed: Dec. 29, 2015

Related U.S. Application Data
(60) Provisional application No. 62/102,081, filed on Jan. 11, 2015.

(51) Int. Cl. A61F 7/02

(52) U.S. Cl. CPC A61F 7/02; A61F 2007/0219; A61F 2007/108

(57) ABSTRACT

A therapy gel pack designed to provide maximum & simultaneous relief for the various contours of the body; which is unavailable in the market today.

The present invention not only provides relief to specific body parts/areas but can also provide it simultaneously (for example: simultaneous relief to shoulder, calf muscle and eye areas).

The present invention is a single product made up of 6 (six) individual therapy packs fastened together via a reseal able locking mechanism.

### TECHNICAL FIELD

This invention relates generally to therapeutic packs providing heat or cold to a body area. It relates more particularly to such hot/cold therapy packs that are truly multipurpose & designed to be applied simultaneously to specific body areas.

### BACKGROUND OF THE INVENTION

Cold therapy, as defined, is the treatment in which all or part of the body is subjected to cold temperatures to relieve pain and increase blood flow.

Hot/Heat Therapy, as defined, may be used, in other circumstances, to warm up or limber muscles by increasing blood flow.

Hot/Cold Therapy packs have been around for several decades. Therapy packs can be applied to almost any body part/area for relief. Most commercial therapy packs available today, are designed to provide relief for 1 (one) body part/area only. For, example: neck, back, shoulder, knee, face etc.

Although some therapy packs can be used to provide relief to various body parts/areas, its design & application may not be best suited to more than one body part/area. For example: a square/rectangle designed gel pack would be better suited for application on the back than on the knee. Similarly, a small round design gel pack would be better suited for application around the eye area than on the shoulder.

In addition, if multiple body parts/areas require simultaneous relief, more than one therapy pack product will be required. For example: a sore shoulder and a hurting knee will require two therapy packs. One each for the shoulder and knee.

### SUMMARY OF THE INVENTION

The present invention overcomes the shortcomings listed above by providing a novel and improved design.

The present invention not only provides relief to specific body parts/areas but can also provide it simultaneously (for example: simultaneous relief to shoulder, calf muscle and eye areas).

The present invention is a single product made up of 6 (six) individual therapy packs fastened together via a reseal able locking mechanism. The individual therapy packs are designed to provide relief to specific body parts/areas. When disassembled (see FIG. 2), the invention can be used simultaneously to provide relief to multiple body areas/parts. When the therapy packs are assembled (see FIG. 1) it becomes a large therapy pack to be applied to a larger area.

### BRIEF DESCRIPTION OF DRAWINGS

FIG. 1

Illustrates an overall visual of the product. This drawing consists of 6 (six) individual therapy packs fastened together which complete the product. The 6 (six) labeled therapy packs are individual therapy packs fastened together via a reseal able fastening (either ‘hook and loop fastener (‘velcro’) or zip locking) mechanism that enables the individual packs to disassemble and re-assemble back into one large therapy pack. The overall size and dimensions/design of the therapy pack are scalable to fit different body types.

The 6 (six) individual therapy packs are designed for a particular body part(s). When fastened together, it forms a large therapy pack that can be used for a large body part/area (example: back or chest)

FIG. 2

Illustrates the individual 6 (six) therapy packs labeled 1 thru 6. The dimensions are alongside each side. The dotted line alongside each of the six therapy packs are indicative of an overlapping bleed area (½ inch). Each of the therapy packs will be fastened to each other via a reseal able locking mechanism located in this (½ inch) bleed area.

When the therapy pack is unfastened, each unfastened therapy pack can be applied individually on a body area/part. Each of the therapy packs are designed to focus and provide relief to a certain area.

### DETAILED DESCRIPTION OF DRAWINGS

The invention is designed to be a single product offering simultaneous therapy (hot/cold) relief to multiple body parts/areas via specifically designed gel packs for each body part(s).

Most products available today, in this category, are designed for use and application on a single body area/part. Although, some can be applied in more than one area, but they are not as effective and cannot be used simultaneously in more than one affected area/body part.

The invention is a single product made up of 6 individual therapy packs fastened together via a reseal able locking mechanism. The therapy packs when disassembled can be used simultaneously to provide relief to multiple body areas/parts. When the therapy packs are assembled it becomes a large therapy pack to be applied to a larger area (example: back, chest area).

The reseal able locking mechanism is made of a reseal able plastic lock or ‘hook and loop fastener (‘velcro’). Once assembled, all the individual pieces will be packaged as one product and will be foldable for packaging purposes.

The gel inside the therapy packs is a soft gel. The chemical composition of the gel will allow it to be flexible upon heating or freezing. Color of the gel can be any color.

The therapy pack can be heated in a microwave or by immersing in hot water. The therapy pack can be frozen in a freezer.

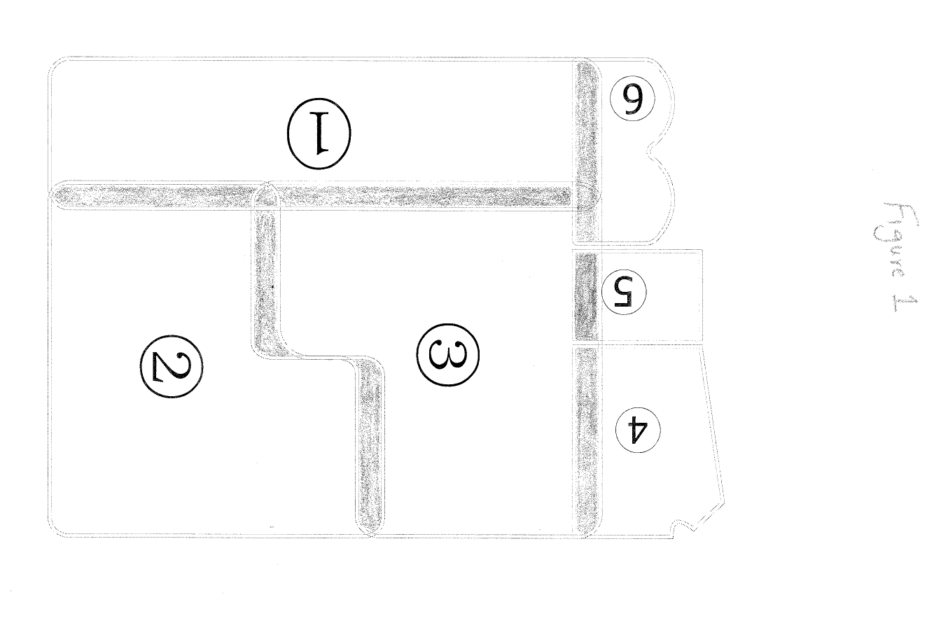

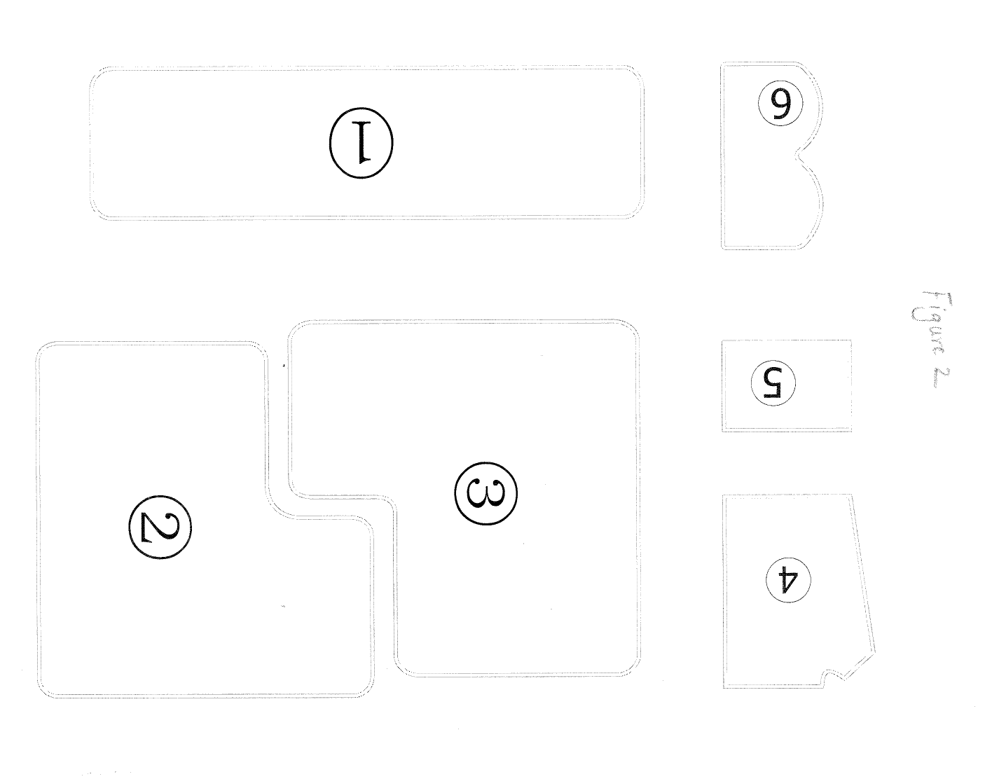

FIG. 1

This figure illustrates the (6) therapy packs assembled together. The shaded area shown in the figure illustrates the overlapping area for each therapy pack. The shaded area is where the packs will be fastened to each other via a reseal able plastic lock or ‘hook and loop fastener (‘velcro’).

FIG. 2

Therapy Pack—1: This is a flat rectangular shaped gel pack. This can be used for several areas including knee, thigh, arms etc. There is a ½ inch bleed on all sides.

Therapy Pack—2: This is a flat unique shaped gel pack. This is used for the right shoulder area. There is a ½ inch bleed on all sides.

Therapy Pack—3: This is a flat unique shaped gel pack. This is used for the left shoulder area. There is a ½ inch bleed on all sides.

Therapy Pack—4: This is a hollow unique shaped gel pack. This is used for the palm/wrist/back of palm area. There is a ½ inch bleed on 2 sides. Hollow will be 0.5″ all around.

Therapy Pack—5: This is a hollow rectangle shaped gel pack. This is used for the finger area. There is a ½ inch bleed on 3 sides. Hollow will be 0.5″ all around

Therapy Pack—6: This is a flat unique shaped gel pack. This is used for the eye area. There is a ½ inch bleed on 1 side. The flat gel packs will be 0.5″ thick.

### CONCLUSION

This invention revolutionizes current therapy packs in the market today. The unique design of this invention not only maximizes relief where applied, but can also do it simultaneously to more than one affected area. In all, a consumer will not need to buy another hot/cold therapy pack product again.

What is claimed is:

1. A multi-purpose gel therapy pack designed to provide relief to specific body areas/parts—a therapy pack uniquely contoured to provide maximum hot/cold relief for each body part.

2. A multi-purpose gel therapy pack as in claim I providing simultaneous relief to more than one body area/part

3. A multi-purpose gel therapy pack as in claim 1 wherein it comprises only the use of one product for multiple body area/part applications
